(19) United States
(12) Patent Application Publication
(10) Pub. No.: US 2019/0346440 A1
(43) Pub. Date: Nov. 14, 2019

(54) HEPARIN-INDUCED THROMBOCYTOPENIA ASSAY

(71) Applicant: BIOKIT RESEARCH & DEVELOPMENTS, S.L.U., Barcelona (ES)

(72) Inventors: Andreas Greinacher, Süderholz (DE); Thi Huong Nguyen, Greifswald (DE)

(73) Assignee: BIOKIT RESEARCH & DEVELOPMENTS, S.L.U., Barcelona (ES)

(21) Appl. No.: 16/387,727

(22) Filed: Apr. 18, 2019

Related U.S. Application Data
(60) Provisional application No. 62/660,589, filed on Apr. 20, 2018.

(51) Int. Cl. G01N 33/564

(52) U.S. Cl. CPC G01N 33/564; G01N 2446/20

(57) ABSTRACT

Disclosed herein are methods and kits for identifying platelet activating anti-PF4/P antibodies in a biological sample useful in diagnosing heparin-induced thrombocytopenia (HIT).

### CROSS-REFERENCE TO RELATED APPLICATIONS

This application claims priority to U.S. Provisional Patent Application No. 62/660,589, entitled HEPARIN-INDUCED THROMBOCYTOPENIA ASSAY filed Apr. 20, 2018, which is incorporated herein by reference in its entirety and for all purposes.

### BACKGROUND

Binding of the chemokine platelet factor 4 (PF4) to polyanions (P) results in multimolecular PF4/P complexes and conformational changes of PF4 that expose neoepitope(s). This triggers an immune response to PF4/P complexes and formation of anti-PF4/P antibodies (Abs). These Abs can induce one of the most frequent immune-mediated adverse drug reactions: heparin-induced thrombocytopenia (HIT). Immunocomplexes composed of anti-PF4/P Abs and PF4/heparin complexes (PF4/H) induce platelet activation via cross-linking FcγRIIa receptors, and also bind to the surface of endothelial cells and monocytes, inducing procoagulant activity.

There is a continuum of clinical sequelae resulting from anti-PF4/P Abs: the majority of individuals remain asymptomatic, while in HIT, anti-PF4/P Abs activate platelets and the clotting system, resulting in paradoxical thrombotic complications during heparin exposure. The most serious presentation is autoimmune HIT, in which antibodies activate platelets in the absence of heparin and can induce spontaneous thrombotic complications.

Although anti-PF4/P Abs bind effectively to immobilized PF4/P complexes in PF4/H enzyme immunosorbent assays (EIAs), only some of them activate platelets in functional assays such as the heparin-induced platelet activation assay (HIPA) or the serotonin release assay (SRA). Accordingly, three groups of anti-PF4/P Abs are distinguished (all are positive in the PF4/H EIA): group-1 Abs do not activate platelets (HIPA negative), group-2 Abs are HIPA positive but only in the presence of heparin, and group-3 Abs activate platelets even in the absence of heparin. A major clinical dilemma is that widely available antigen diagnostic test systems cannot differentiate between these 3 groups of antibodies, which makes them only meaningful for exclusion of HIT in the case of a negative result. Accordingly, there remains a need for a diagnostic assay that can differentiate between clinically relevant (i.e., platelet activating) and non-clinically relevant (i.e., non-platelet activating) anti-PF4/P antibodies.

### SUMMARY

Heparin is one of the most frequently used drugs in hospitals. Therefore, heparin-induced thrombocytopenia (HIT) is one of the most frequent severe adverse drug effects in clinical medicine, mediated by anti-platelet factor 4 (PF4)/polyanion antibodies (anti-PF4/P Abs). However, less than 50% of anti-PF4/P Abs activate platelets, and only those are clinically relevant. The inability of currently available antigen assays to differentiate between clinically relevant, platelet activating, and clinically irrelevant, non-activating anti-PF4/P Abs causes substantial over-diagnosis of HIT, resulting in adverse effects due to overtreatment. Here, it was identified by single-molecule force spectroscopy, that only clinically relevant anti-PF4/P Abs increase their binding force to platelets strongly in the presence of PF4, reaching a maximum at 50 μg PF4/mL. This physical characteristic of clinically relevant, platelet activating anti-PF4/P antibodies provides the basis for an accurate and reproducible assay for diagnosing HIT.

One aspect of the present invention is directed to a method of identifying platelet activating anti-PF4/P antibodies in a biological sample involving the steps of a) providing PF4-coated or PF4/P-coated biological membranes or PF4-coated or PF4/P coated artificial membranes; b) contacting the PF4-coated or PF4/H-coated membranes with a biological sample from a patient in the presence of conditions whereby platelet-activating antibodies bind the coated membranes and non-activating antibodies do not bind the coated membranes; and c) detecting antibodies bound to the coated membranes.

In some embodiments, the biological membranes are whole cells, preferably platelets. In some embodiments, the biological membranes are cell fractions, preferably platelet fractions. In certain embodiments, the platelet fractions are platelet microparticles.

In some embodiments, the biological or artificial membranes are coated onto a solid substrate.

In some embodiments, the artificial membranes are phospholipids and PF4-binding molecules and optionally heparin-binding molecules. In some embodiments, the PF4-binding molecules and heparin-binding molecules are proteins, carbohydrates, nucleic acids or polymers. Representative examples of proteins include PF4 antibodies or fragments thereof, heparin antibodies or fragments thereof, FcγRIIa receptors or fragments thereof, or protamines or fragments thereof. Representative examples of nucleic acids include affimers that specifically bind PF4 or heparin. Representative examples of polymers include polyanionic polymers, such as polyvinyl sulfate, polystyrene sulfate, chondroitin sulfate, dermatan sulfate, keratan sulfate, heparan sulfate and heparin.

In some embodiments, the conditions include pH and ion concentration. In some embodiments, the method further includes a step between steps b) and c) of washing the membranes with a solution comprising a pH and ion concentration such that platelet-activating antibodies remain bound and non-activating antibodies are removed. In certain embodiments, the pH is about 6.0 and the ion concentration is about 50 mM NaCl.

In some embodiments, the PF4-coated membranes are formed by incubating with 2-500 μg/ml PF4, preferably with 25-75 μg/ml PF4. In certain embodiments, the incubating is at 37° C. for about 30 minutes.

In some embodiments, the PF4/P-coated membranes are formed by incubating with 2-500 μg/ml of PF4/P pre-formed by PF4 and a polyanionic polymer. Representative examples of polyanionic polymers include polyvinyl sulfate, polystyrene sulfate, chondroitin sulfate, dermatan sulfate, keratan sulfate, heparan sulfate and heparin. In certain embodiments, the polyanionic polymer is polyvinyl sulfate. In other embodiments, the polyanionic polymer is heparin. In certain embodiments, the incubating is at 37° C. for about 30 minutes.

In some embodiments, the solid substrate is glass surfaces, plastic surfaces, silicon surfaces, solid organic polymers, cellulose/cellulose-based membranes, colloidal metal particles or magnetic particles. In certain embodiments, the solid substrate is magnetic particles. In some embodiments, the solid substrate is a glass surface which is a glass slide. In some embodiments, the solid substrate is a plastic surface which is a microtiter plate or a well thereof. In some embodiments, the solid substrate is a colloidal metal particle which is a gold particle. In yet other embodiments, the solid substrate is a solid organic polymer which is a latex bead.

In some embodiments, the biological sample is a blood sample, a serum sample or a plasma sample, preferably a serum sample.

In some embodiments, the detecting is carried out by an assay such as an enzyme linked immunosorbent assay (ELISA), a radioimmunoassay (MA), an immuno radiometric assay (IRMA), a fluorescent immunoassay (FIA), a chemiluminescent immunoassay (CLIA), an electro-chemiluminescent immunoassay (ECL) or an agglutination assay.

Another aspect of the present invention is directed to a method of identifying platelet activating anti-PF4/P antibodies in a biological sample including the steps of: a) providing biological membranes or artificial membranes; b) contacting the membranes with a first portion of a biological sample from a patient; c) detecting antibodies bound to the membranes; d) optionally, measuring binding force of the antibodies bound to the membranes; e) providing PF4-coated or PF4/P-coated biological membranes or PF4-coated or PF4/P-coated artificial membranes; f) contacting the coated membranes with a second portion of a biological sample from said patient; g) detecting antibodies bound to the coated membranes; and h) optionally, measuring binding force of the antibodies bound to the coated membranes; wherein a higher number of bound antibodies in g) than in c) indicates presence of platelet activating anti-PF4/P antibodies in said biological sample or wherein a binding force measured in h) greater than a binding force measured in d) indicates presence of platelet activating anti-PF4/P antibodies in said biological sample.

In some embodiments, the biological membranes are whole cells, preferably platelets. In some embodiments, the biological membranes are cell fractions, preferably platelet fractions. In certain embodiments, the platelet fractions are platelet microparticles.

In some embodiments, the biological or artificial membranes are coated onto a solid substrate.

In some embodiments, the artificial membranes are phospholipids and PF4-binding molecules and optionally heparin-binding molecules. In some embodiments, the PF4-binding molecules and heparin-binding molecules are proteins, carbohydrates, nucleic acids or polymers. Representative examples of proteins include PF4 antibodies or fragments thereof, heparin antibodies or fragments thereof, FcγRIIa receptors or fragments thereof, or protamines or fragments thereof. Representative examples of nucleic acids include affimers that specifically bind PF4 or heparin. Representative examples of polymers include polyanionic polymers, such as polyvinyl sulfate, polystyrene sulfate, chondroitin sulfate, dermatan sulfate, keratan sulfate, heparan sulfate and heparin.

In some embodiments, the method further includes a step between steps b) and c) and between steps f) and g) of washing the membranes with a solution comprising a pH and ion concentration such that platelet-activating antibodies remain bound and non-activating antibodies are removed. In some embodiments, the pH is about 6.0 and the ion concentration is about 50 mM NaCl.

In some embodiments, the PF4-coated membranes are formed by incubating with 2-500 μg/ml PF4, preferably with 25-75 μg/ml PF4. In certain embodiments, the incubating is at 37° C. for about 30 minutes.

In some embodiments, the PF4/P-coated membranes are formed by incubating with 2-500 μg/ml of PF4/P pre-formed by PF4 and a polyanionic polymer. Representative examples of polyanionic polymers include polyvinyl sulfate, polystyrene sulfate, chondroitin sulfate, dermatan sulfate, keratan sulfate, heparan sulfate and heparin. In certain embodiments, the polyanionic polymer is polyvinyl sulfate. In other embodiments, the polyanionic polymer is heparin. In certain embodiments, the incubating is at 37° C. for about 30 minutes.

In some embodiments, the solid substrate is glass surfaces, plastic surfaces, silicon surfaces, solid organic polymers, cellulose/cellulose-based membranes, colloidal metal particles or magnetic particles. In certain embodiments, the solid substrate is magnetic particles. In some embodiments, the solid substrate is a glass surface which is a glass slide. In some embodiments, the solid substrate is a plastic surface which is a microtiter plate or a well thereof. In some embodiments, the solid substrate is a colloidal metal particle which is a gold particle. In yet other embodiments, the solid substrate is a solid organic polymer which is a latex bead.

In some embodiments, the biological sample is a blood sample, a serum sample or a plasma sample, preferably a serum sample.

In some embodiments, the detecting is carried out by an assay such as an enzyme linked immunosorbent assay (ELISA), a radioimmunoassay (MA), an immuno radiometric assay (IRMA), a fluorescent immunoassay (FIA), a chemiluminescent immunoassay (CLIA), an electro-chemiluminescent immunoassay (ECL) or an agglutination assay.

In some embodiments, the step of measuring is carried out by single-molecule force spectroscopy (SMFS) or atomic force microscopy (AFM).

Another aspect of the present invention is directed to kits including: a) a solid substrate coated with PF4-coated or PF4/P-coated membranes.

In some embodiments, the coated membranes are biological membranes or artificial membranes.

In some embodiments, the biological membranes are whole cells, preferably platelets. In some embodiments, the biological membranes are cell fractions, preferably platelet fractions. In certain embodiments, the platelet fractions are platelet microparticles.

In some embodiments, the artificial membranes are phospholipids and PF4-binding molecules and optionally heparin-binding molecules. In some embodiments, the PF4-binding molecules and heparin-binding molecules are proteins, carbohydrates, nucleic acids or polymers. Representative examples of proteins include PF4 antibodies or fragments thereof, heparin antibodies or fragments thereof, FcγRIIa receptors or fragments thereof, or protamines or fragments thereof. Representative examples of nucleic acids include affimers that specifically bind PF4 or heparin. Representative examples of polymers include polyanionic polymers, such as polyvinyl sulfate, polystyrene sulfate, chondroitin sulfate, dermatan sulfate, keratan sulfate, heparan sulfate and heparin.

In some embodiments, the kit further includes: b) a washing solution having a pH and ion concentration such that platelet-activating antibodies remain bound and non-activating antibodies are removed. In certain embodiments, the pH is about 6.0 and the ion concentration is about 50 mM NaCl.

In some embodiments, the PF4-coated membranes are formed by incubating with 2-500 μg/ml PF4, preferably with 25-75 μg/ml PF4. In certain embodiments, the incubating is at 37° C. for about 30 minutes.

In some embodiments, the PF4/P-coated membranes are formed by incubating with 2-500 μg/ml of PF4/P pre-formed by PF4 and a polyanionic polymer. Representative examples of polyanionic polymers include polyvinyl sulfate, polystyrene sulfate, chondroitin sulfate, dermatan sulfate, keratan sulfate, heparan sulfate and heparin. In certain embodiments, the polyanionic polymer is polyvinyl sulfate. In other embodiments, the polyanionic polymer is heparin. In certain embodiments, the incubating is at 37° C. for about 30 minutes.

In some embodiments, the solid substrate is glass surfaces, plastic surfaces, silicon surfaces, solid organic polymers, cellulose/cellulose-based membranes, colloidal metal particles or magnetic particles. In certain embodiments, the solid substrate is magnetic particles. In some embodiments, the solid substrate is a glass surface which is a glass slide. In some embodiments, the solid substrate is a plastic surface which is a microtiter plate or a well thereof. In some embodiments, the solid substrate is a colloidal metal particle which is a gold particle. In yet other embodiments, the solid substrate is a solid organic polymer which is a latex bead.

In some embodiments, the kit further includes: c) appropriate reaction buffers for performing an assay selected from the group consisting of: an enzyme linked immunosorbent assay (ELISA), a radioimmunoassay (RIA), an immuno radiometric assay (IRMA), a fluorescent immunoassay (FIA), a chemiluminescent immunoassay (CLIA), an electro-chemiluminescent immunoassay (ECL) and an agglutination assay.

In yet other embodiments, the kit further includes: d) a positive control; and e) a negative control. In some embodiments, the positive control is a solution containing a known concentration of platelet activating anti-PF4/P antibodies. In some embodiments, the negative control is a solution containing no anti-PF4/P antibodies or a known concentration of non-activating anti-PF4/P antibodies.

### BRIEF DESCRIPTION OF THE DRAWINGS

FIGS. 1A-C are a diagram (A) and graphs (B and C) showing pre-selection of specific antibodies in each group. (A) A single covalently immobilized antibody on an AFM-tip is brought into contact with PF4/H complexes coated on the substrate for interaction and the binding force is recorded when the tip moves away from the substrate. (B) Individual dots present the mean of the binding force of a single antibody immobilized on a cantilever (n=3 sera for each group). Group-1 Abs (dark cyan, 43 cantilevers) show homogeneous binding forces <60 pN comparable to KKO (gray, 18 cantilevers), whereas group-2 (blue, 54 cantilevers) and group-3 (red, 51 cantilevers) contain antibodies with a large variation of binding strengths (up to ˜150 pN). (C) Binding strengths of the selected anti-PF4/P Abs coated on the cantilevers used for interacting with platelets (3 cantilevers from each serum, n=3 sera, total 9 cantilevers per group).

FIGS. 2A-C are a diagram (A) and graphs (B and C) showing binding forces of anti-PF4/P Abs to washed platelets. (A) A single antibody was immobilized covalently on an AFM-cantilever tip, while platelets were immobilized on collagen-G coated glass. (B) A representative example of binding forces of individual antibodies: group-3 Abs (red) show high rupture forces with two distinguishable peaks (1^{st} and 2^{nd} peak) while KKO (gray), group-1 (dark cyan), and group-2 Abs (blue) show rupture forces similar to human control IgG (black). (C) Average rupture forces and corresponding SD of each cantilever determined by Gaussian fits. Only group-3 Abs display substantial binding to washed platelets and show two distributions of forces at <300 pN (1^{st} peak) and at >300 pN (2^{nd} peak).

FIGS. 3A-F are a diagram (A) and graphs (B-F) showing the interaction of anti-PF4/P Abs with PF4 coated platelets. (A) Platelets were coated with different PF4 concentrations (0-100 μg/ml) and the Abs on the cantilevers were brought into contact with the platelet surfaces for interaction. (B) control IgG and (C) group-1 Abs showed weak interactions mostly <300 pN, while (D) KKO and (E) group-2 Abs interacted stronger at 25- and 50 μg/ml PF4 compared to that at 0- or 100 μg/ml PF4. (F) Group-3 Abs interacted strongly with platelets at all PF4 concentrations with more counts at higher binding forces when PF4 was added. The vertical red line shows 300 pN the maximal binding forces of control IgG for comparison.

FIGS. 4A-E are box plots summarizing the binding forces of anti-PF4/P Abs to platelets coated with different PF4 concentrations. For each group, antibodies purified from 3 different sera were used and 3 different cantilevers were tested per antibody fraction purified from each serum. About 150-300 specific interactions were obtained when each antibody immobilized on the tip interacts with platelets at a specific PF4 concentration. (A) Control IgG and (B) group-1 Abs show binding forces lower than 300 pN (red dotted lines), which did not change substantially at different PF4 concentrations, whereas (C) KKO, (D) group-2 and (E) group-3 Abs showed higher binding forces, which peaked at a PF4 concentration of 50 μg/mL (50-75 μg/mL for KKO).

FIGS. 5A-C are graphs showing binding strength of anti-PF4/P Abs with PF4/H complexes coated platelets. (A) When PF4/H complexes were coated on platelets, group-2 (blue) and group-3 (red) Abs and KKO (gray) showed much higher binding forces than group-1 Abs (dark cyan) and control IgG (black) which show rupture forces <300 pN (red line). (B) The binding forces did not differ largely for all antibodies between PF4/H (red) or 50 μg/ml PF4 coated platelets (dashed-black). Panel (C) shows the comparison of the frequency of interactions (count number) of Abs when interacting with 50 μg/ml PF4- (dashed-black) and PF4/H complex (red) coated platelets.

FIGS. 6A-G are diagrams (A and B) and graphs (C-G) showing the comparison of binding strengths of the same anti-PF4/P Abs to PF4/H complexes coated on (A) the solid phase and on (B) platelets. (C) The binding forces F of Abs to PF4/H complexes coated on the solid phase are much lower than when they were coated on platelets (data show average of the forces from three different antibodies obtained from each serum [n=3 sera/group]). (D) Representative force-distance curve of a group-3 Abs from of PF4/H complexes coated to the solid phase showing one rupture event only. (E, F) In contrast, the rupture force pattern was much more complex when antibodies bound to platelets coated with PF4. These curves consist of different types of forces (F₁ and F₂), which contribute to the final rupture forces F(F=F₁+F₂). F₂ represents the final rupture force of the antibody from its antigen, while F₁ is a composite of multiple interactions of the antibody with the platelet surface (e.g. stretching of the component(s) to which PF4 has bound and also the cell membrane). (E) control IgG shows only a minimal rupture force F₂. Group-1 Abs display much lower forces (both F₁ and F₂) than KKO (F) or group-2 or 3 Abs (which show similar curves as KKO (FIG. 11). (G) The total rupture forces (F, black) when Abs interact with PF4/H complexes immobilized on the solid phase are significantly lower than the F₂ component of the rupture force (red) representing the binding forces between Abs and PF4/P complexes on the platelet surface.

FIGS. 7A-F are schematics of a model of anti-PF4/H Abs binding when different concentrations of PF4 are coated on platelets. (A) The distance between the two binding sites of two IgG Fab arms is ˜15 nm, which is approximately equal to the size of three PF4 molecules (5 nm diameter per PF4 tetramer) aligned continuously on a surface. (B) Without additional PF4, few available PF4s bind to GAGs on platelets which allow binding of group-3 but not of group-2 Abs as no PF4/P complexes are formed. (C) At low added PF4 concentrations, only some large complexes were formed with ≥3 PF4 molecules which result in few optimal binding sites for group-2 Abs whereas complexes composed of <3 PF4 molecules allow binding of only one Fab-arm leading to weak binding forces. (D) At 50 μg/ml, optimal binding of PF4 molecules to platelet GAGs is achieved and optimal antibody binding epitopes are induced. (E) At 100 μg/ml, large and heterogenous complexes are formed, as several PF4 molecules compete for the negative charges on the platelet surface. This partially blocks optimal antibody binding epitopes. (F) Pre-formation of PF4/H complexes results in large multimolecular complexes, which expose optimal binding epitopes for PF4/P Abs binding. Of note, multimolecular complexes between PF4 and polyanions can much easier be formed in the fluid phase, when both binding partners are fully flexible as compared to the platelet surface, where polyanions such as chondroitin sulfate are fixed.

FIGS. 8A-D are graphs of the amount of PF4 bound to the platelet surface. (A-C) PF4 binding to platelets increases dose-dependently. (D) In the presence of high heparin concentrations, PF4 molecules are displaced from the platelet surface which results in low binding.

FIGS. 9A-E are plots showing the binding force of individual anti-PF4/P Abs on platelets coated with different PF4 concentrations. At every concentrations, (A) control IgG and (B) group-1 Abs showed weak interactions mostly <300 pN (red line), while (C) KKO and (D) group-2 Abs interacted stronger at concentrations between 25- to ˜50 μg/ml PF4 compared to platelets precoated with 0- or 100 μg/ml PF4. (E) Group-3 Abs interacted strongly with platelets at all PF4 concentrations.

FIGS. 10A and B are box plots of binding strength of anti-PF4/P Abs with PF4/H complexes coated platelets. In the presence of high concentrations of heparin (100 IU/ml), both interaction force (A) and interaction counts (B) significantly decreased for KKO and group-2 Abs but not for control IgG, group-1, and group-3 Abs.

FIGS. 11A and B are graphs of typical force-distance curves of the interactions between Abs and platelets coated with 50 μg/ml PF4. The force F₂ represents the binding forces between the antibody and PF4/P antigens obtained by group-2 Abs (A) and group-3 (B) Abs.

### DETAILED DESCRIPTION

Previously, it was illustrated that the binding characteristics of anti-PF4/P Abs are associated with their biological activity, i.e. group-1 Abs bind relatively weakly to PF4/H complexes, group-2 Abs bind stronger, and group-3 Abs bind strongest not only to PF4/H complexes but also to PF4 alone. Recently, it was found that KKO, a monoclonal antibody, which mimics platelet activating human anti-PF4/P Abs, bound weakly to PF4/H complexes on a solid phase. This was unexpected, as usually antibodies with platelet activating capacity show higher binding forces. However, when KKO interacted with PF4 pre-coated platelets, its binding forces enhanced about 4-fold. This indicated that the presentation of PF4/H complexes in these two systems may differ.

Preincubation of platelets with PF4 enhances the sensitivity of platelet activation assays for anti-PF4/P Abs as compared with non-coated platelets. In addition, platelet-derived polyanions such as chondroitin sulfate and polyphosphates interact with PF4, inducing a conformational change which allows binding of anti-PF4/P Abs. Together, these studies indicate that anti-PF4/P Abs can bind to PF4 coated platelets and probably interact differently with PF4/H complexes in a purified system as compared to PF4/P complexes on the platelet surface. Here, the binding forces of human anti-PF4/P Abs to PF4 coated on the platelet surface in the presence and absence of heparin were investigated. It was found that the binding force of platelet activating anti-PF4/P Abs to platelets increased when PF4 or PF4/H complexes were added, while this was not the case for non-platelet activating anti-PF4/P Abs. This observation provides the basis for antigen assays differentiating clinically relevant (i.e., platelet activating) from non-clinically relevant (i.e., non-platelet activating) antibodies.

### Methods Ethics

The use of human sera obtained from healthy volunteers and patients with HIT was approved by the ethics board at the Universitätsmedizin Greifswald.

### Antibody Purification and Characterization

For each group (group-1, group-2, group-3), anti-PF4/P Abs were isolated from three independent sera by two-step affinity chromatography, i.e. first by a protein G column to isolate total IgG and then by a PF4/H column to extract anti-PF4/P Abs as previously described. (Nguyen T H et al., “Anti-platelet factor 4/polyanion antibodies mediate a new mechanism of autoimmunity”, Nature Communications, 2017; 8). Briefly, protein G-coated sepharose beads (GE Healthcare Europe GmbH, Freiburg, Germany) were incubated with human serum for binding of total IgG. Then, total IgG was eluted from the column and transferred to a new column containing beads pre-coated with preformed PF4/H complexes (0.5 IU/ml UFH [Heparin-Natrium-25000 (Ratiopharm, Ulm, Germany] and 20 mg/ml mixture of 30% biotinylated PF4:70% PF4; Chromatec, Greifswald, Germany) in PBS, RT for 1 h for purification of anti-PF4/P Abs. The purity of the affinity-purified Abs eluted from the column was controlled by SDS-PAGE, PF4/H IgG enzyme immunosorbent assay (EIA), and HIPA test as described. (Nguyen T H et al., “Anti-platelet factor 4/polyanion antibodies mediate a new mechanism of autoimmunity”, Nature Communications, 2017; 8). The purified antibodies were dialyzed against PBS pH7.4 before immobilizing on AFM-tips. KKO (BIOZOL, Munchen, Germany) was used as a standard, whereas human IgG purified from sera of healthy volunteers, testing negative in both PF4/H EIA and HIPA, was used as negative control.

### Determination of the Binding Force of Anti-PF4/P Abs by Single-Molecule Force Spectroscopy (SMFS)

Immobilization of Single Antibodies on an Atomic Force Microscopy (AFM)-Tip:

anti-PF4/P Abs were covalently and flexibly immobilized on AFM-tips (6 pN/nm, Olympus Biolever, Tokyo, Japan) as previously described. (Nguyen T H et al., “Anti-platelet factor 4/polyanion antibodies mediate a new mechanism of autoimmunity”, Nature Communications, 2017; 8; Nguyen T H and Greinacher, A, “Effect of pH and ionic strength on the binding strength of anti-PF4/polyanion antibodies”, Eur Biophys J. 2017). A long thiol-PEG-carboxylic acid linker (HS-PEG-COOH, PEG Mw 3400 Da, Nanocs, USA) was used to link the antibody (70 μg/ml) covalently to the tip via gold-thiol and amide bond coupling. After rinsing with PBS, the cantilevers were kept at 4° C. and used within three days.

Immobilization of Platelets on a Solid Substrate:

To avoid signals resulting from binding of the antibody Fc parts, platelet FcγRIIa receptors were blocked by preincubation of washed platelets with the monoclonal antibody IV.3 at 37° C. for 30 min (100 μg/mL final) before coating with PF4 or PF4/H complexes. To coat platelets, PF4 (0-100 μg/ml) or PF4/H complexes (pre-formed by 20 μg/ml PF4; 0.5 IU/ml heparin) were added to the IV.3 pre-coated platelets and incubated at 37° C. for 30 min. To immobilize platelets on a solid substrate, non-PF4-coated-, PF4-, or PF4/H complexes coated platelets were incubated at RT for 10 min on glass slides pre-coated with 20 μg/ml collagen G, 3 h at 37° C. After that, unbound molecules were rinsed away with PBS containing 1.0 mM CaCl₂). The cantilevers were kept at 4° C. and used within three days, whereas platelets on the substrates were used immediately after immobilization.

Measurement of Antibody-Platelet Binding Force:

SMFS measurements were carried out in PBS containing 1.0 mM CaCl₂) using JPK NanoWizard 3 (Berlin, Germany). Before each experiment, cantilever spring constant was independently determined by a thermal tune procedure available at the JPK system. For each experimental condition, 900 force-distance (F-D) curves were recorded at the same loading force 200 pN and tip velocity 1,000 nm/s. The rupture forces at the final rupture points before the cantilevers went back to the rest position were collected using JPK data processing software (version 4.4.18+). Origin software (version 8.6) was used for data analysis and the mean rupture force values and their corresponding errors were determined by applying Gaussian fits to the data.

PF4 Density on Platelet Surfaces:

Washed platelets were incubated with PF4 (0-, 50-, and 100 μg/mL) at 37° C. for 30 min as described. (Krauel K et al., “Heparin-induced thrombocytopenia—therapeutic concentrations of danaparoid, unlike fondaparinux and direct thrombin inhibitors, inhibit formation of platelet factor 4-heparin complexes”, Journal of Thrombosis and Haemostasis, 2008; 6(12):2160-67). Platelets were then fixed with 1% paraformaldehyde (Merck, Darmstadt, Germany) for 20 min, 4° C. and washed twice at 4° C., 600 g, 7 min with buffer (137 mM NaCl, 2.7 mM KCl, 2 mM MgCl₂.6H₂O, 2 mM CaCl₂.2H₂O, 12 mM NaHCO₃, 0.4 mM NaH₂PO₄, 0.4% bovine serum albumin (BSA), 0.1% glucose, pH 7.2). After that, platelets were incubated with rabbit anti-human PF4 (Dianova, Marl, Germany), fluorescein isothiocyanate (FITC)-labeled with the FluoReporter FITC Protein Labeling Kit (Molecular Probes, Eugene, Oreg., USA) for 30 min, 4° C., and washed before measuring by flow cytometry.

### Classification of Anti-PF4/P Antibodies

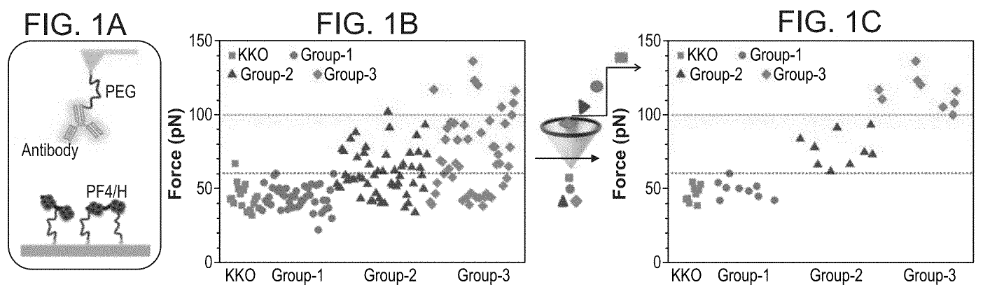

Sera which activate platelets in the presence of heparin contain group-2 Abs with binding forces between 60 and 100 pN to PF4/H complexes immobilized on a glass surface, whereas sera activating platelets without the addition of heparin contain group-3 Abs with binding forces >100 pN. Due to the polyclonal immune response, antibody fractions of sera containing group-2 and group-3 Abs also contain anti-PF4/P Abs with lower binding affinities. Therefore, the binding strength of the antibodies immobilized to the cantilever was predetermined with PF4/H complexes immobilized on a solid phase. For that, cantilevers (43-, 54- and 51 cantilevers for group-1, group-2, and group-3Abs, respectively) were coated with antibodies purified from each serum (n=3) and the binding strengths with PF4/H complexes were measured (FIG. 1A).

Among these, the cantilevers coated with antibodies purified from group-2 sera, which exhibit binding strengths in the range of 60 and 100 pN were selected, and with antibodies purified from group-3 sera, which exhibit binding strengths >100 pN (FIG. 1C). In total nine cantilevers (3 cantilevers per serum, n=3 sera) were selected for each antibody group and nine cantilevers coated with KKO were used as a standard. As all cantilevers coated with control IgG showed only a few interactions with PF4/H complexes in the solid phase, nine random cantilevers coated with these antibodies were used. The preselection procedure also allowed validating that only a single antibody was bound to the cantilever as multiple antibodies typically produce more than one rupture signal which can be recognized in the force-distance curves, as it is extremely unlikely that multiple antibodies bind with the same geometry to the cantilever.

### Results

### Binding Strengths of Anti-PF4/P Abs to Washed Platelets

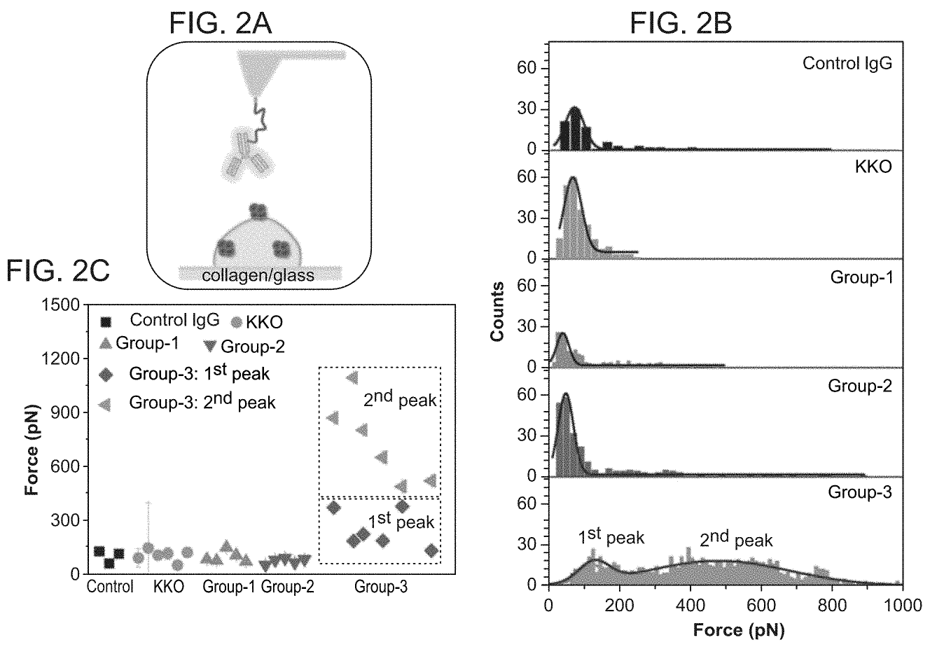

First, the binding strengths of the above defined anti-PF4/P Abs to washed platelets, which express small amounts of PF4 on the surface was determined (FIG. 8). FIG. 2B illustrates representative individual rupture force distributions obtained with each antibody group. Only group-3 Abs showed significantly stronger binding forces than control IgG. Particularly, there are two binding patterns of group-3 Abs, i.e. one giving mean binding forces <300 pN (1^{st} peak) and the other giving mean binding forces >300 pN (2^{nd} peak) (FIG. 2C). The large range of binding forces even with the same antibody indicates that antibodies found only few optimal binding sites on the platelet surface.

### Binding Strengths of Anti-PF4/P Abs to PF4 Coated Platelets

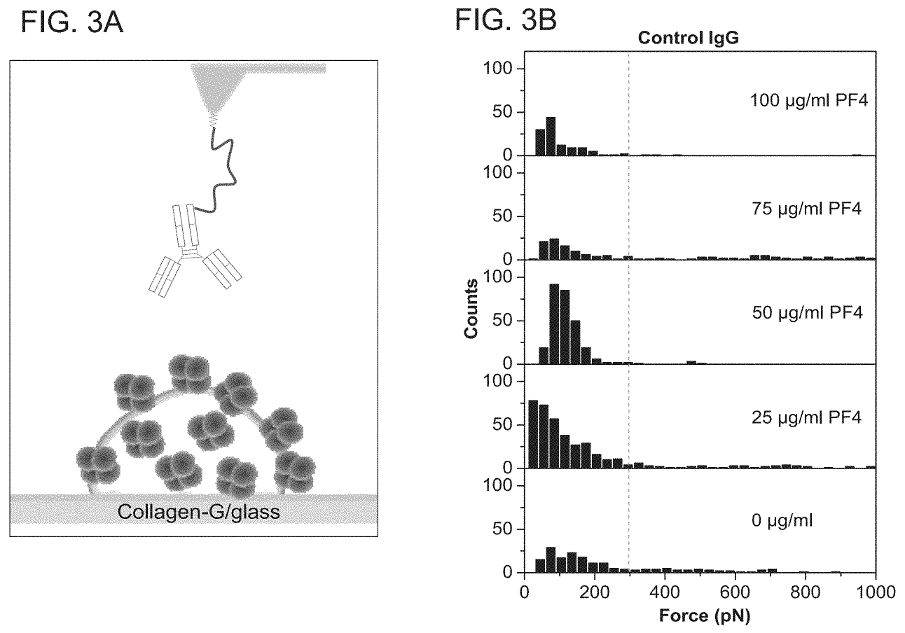

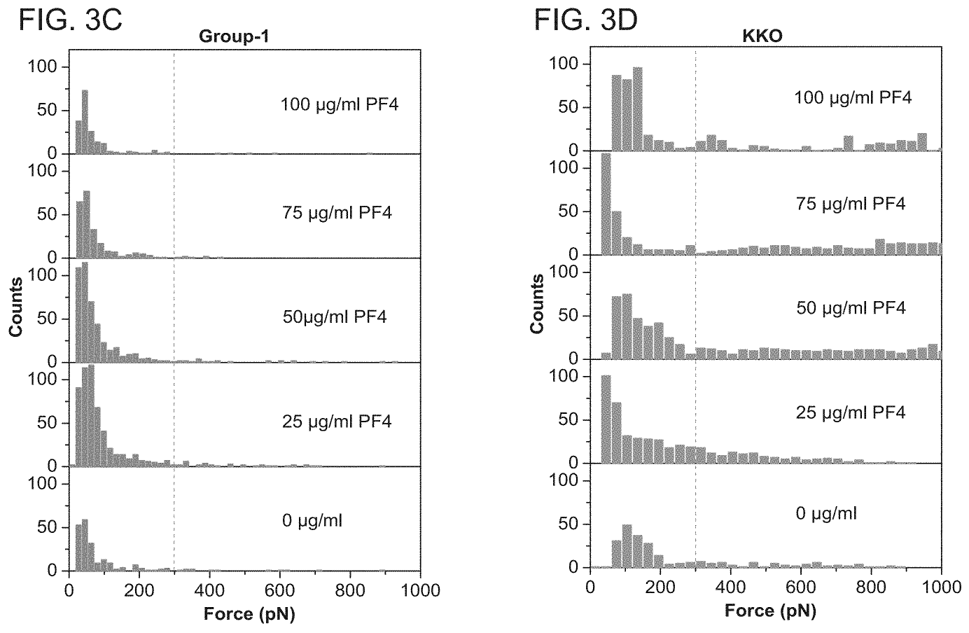

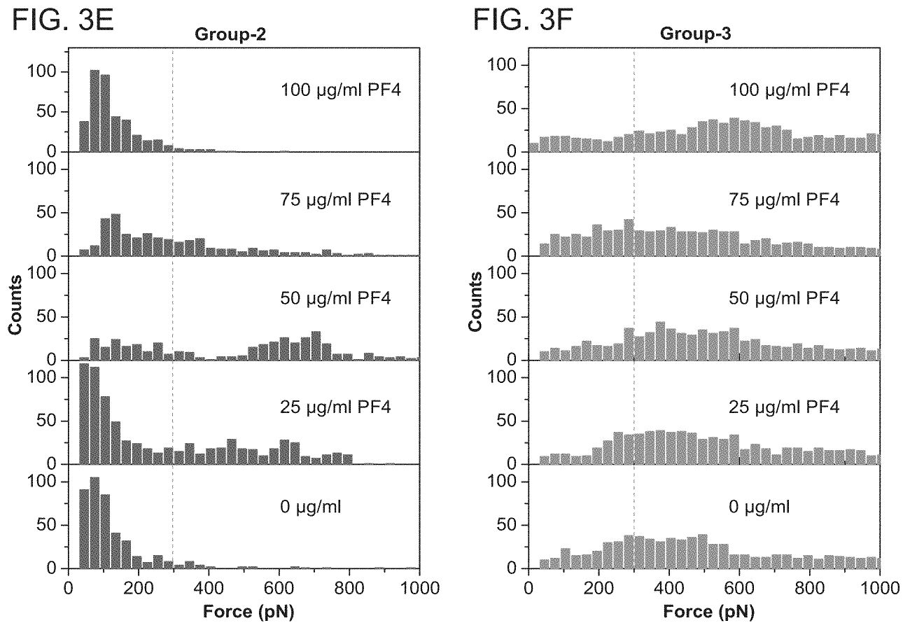

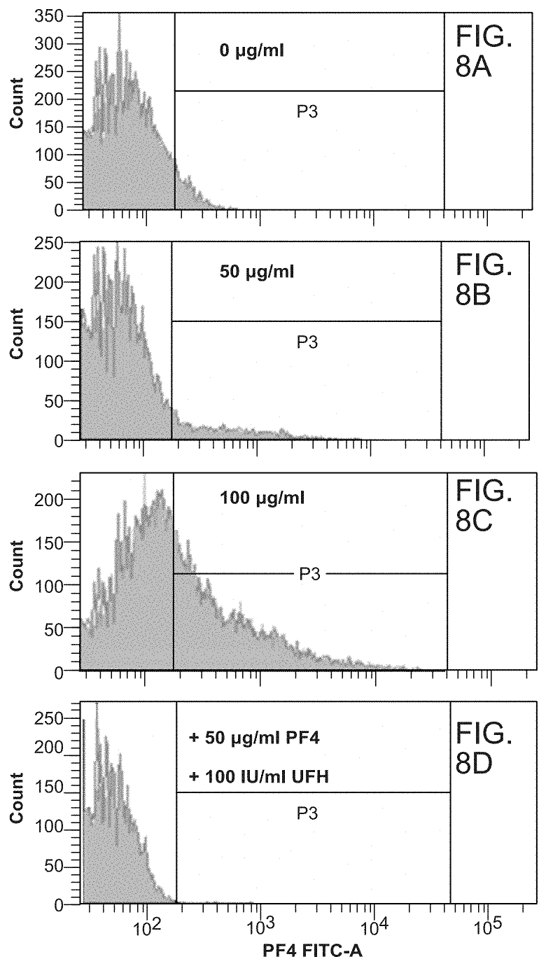

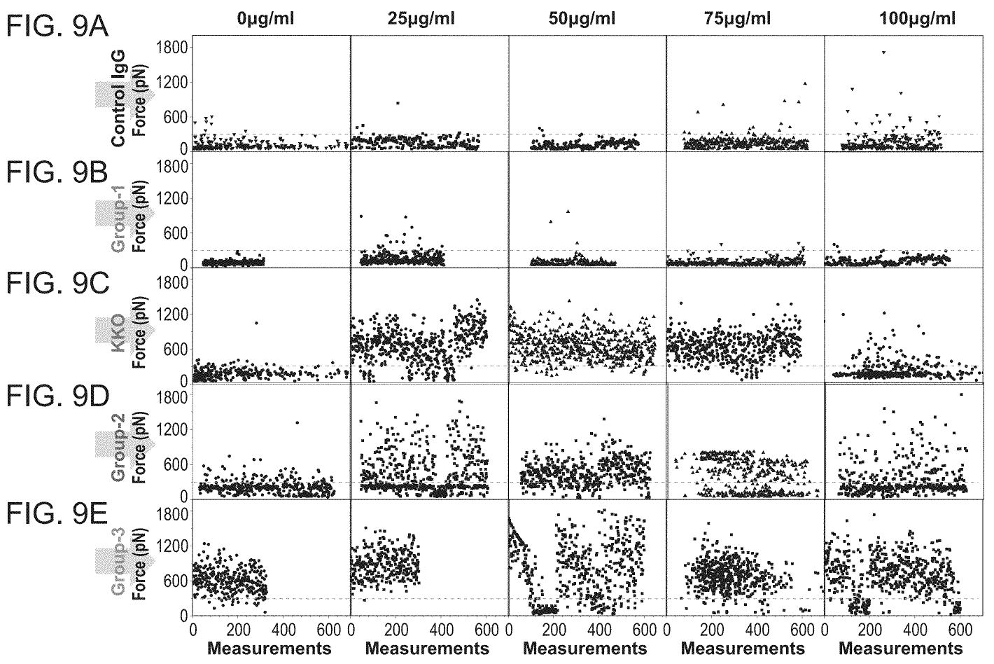

After PF4 coating (FIG. 3A), binding forces of control IgG (FIG. 3B) and group-1 Abs (FIG. 3C) to platelets did not significantly increase. In contrast, binding forces of KKO (FIG. 3D) and group-2 Abs (FIG. 3E) increased with added PF4 in a bell-shaped manner, which peaked at PF4 concentrations of ˜50 μg/ml and then decreased again. The binding forces of group-3 Abs did not show major changes at different PF4 concentrations (FIG. 3F). Individual rupture forces for each antibody group at different PF4 coating concentrations are shown in FIG. 9.

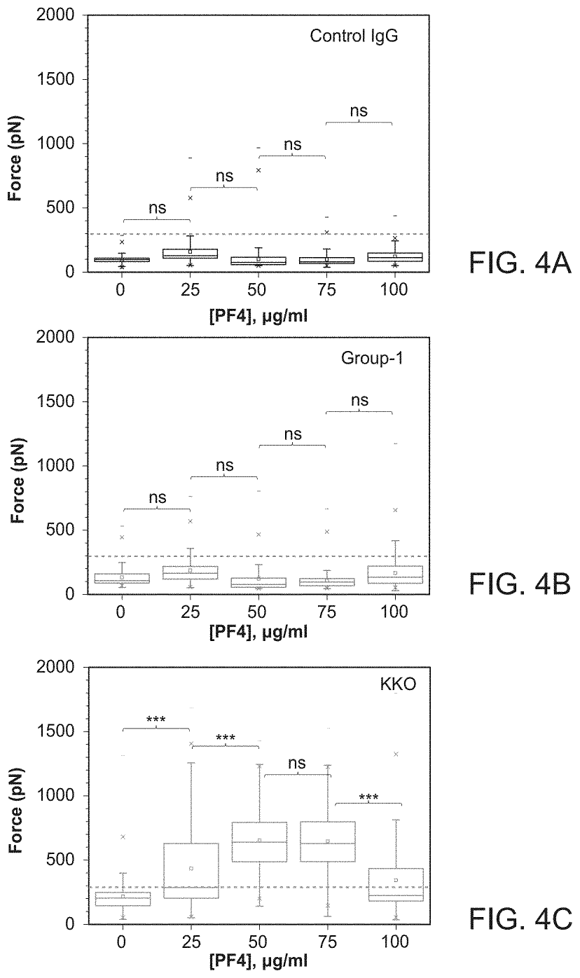

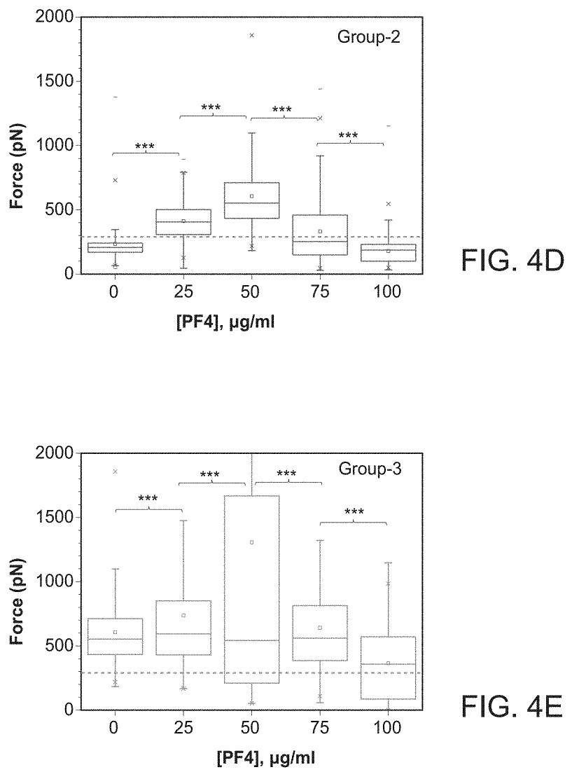

FIG. 4 summarizes the individual binding forces of the different antibodies with PF4 coated platelets obtained in all experiments. At every PF4 concentration, control IgG (FIG. 4A) and group-1 Abs (FIG. 4B) showed low binding forces, which did not change in the presence of PF4, whereas KKO (FIG. 4C), group-2 (FIG. 4D) and group-3 (FIG. 4E) Abs showed much higher binding forces, which reached maximal values at a concentration of 50 μg/ml (KKO at 50 and 75 μg/mL).

### Binding Strengths of Anti-PF4/P Abs to PF4/H Coated Platelets

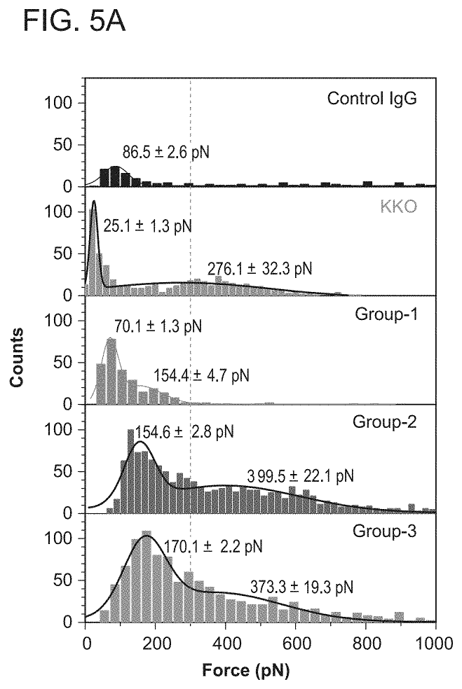

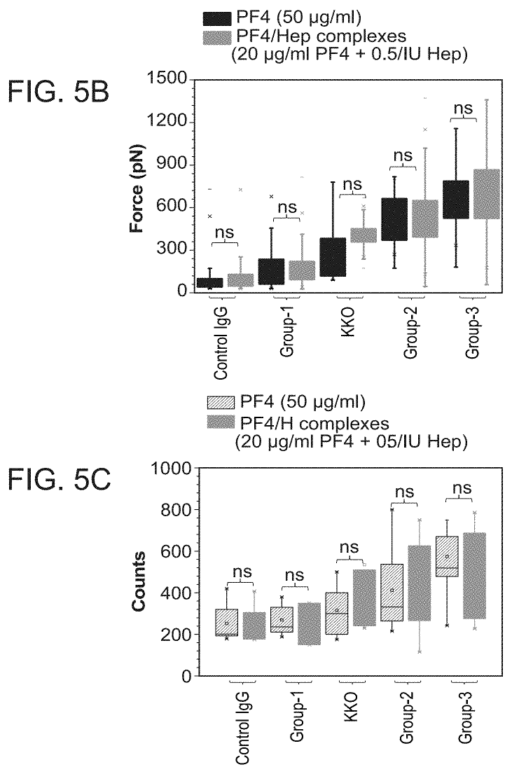

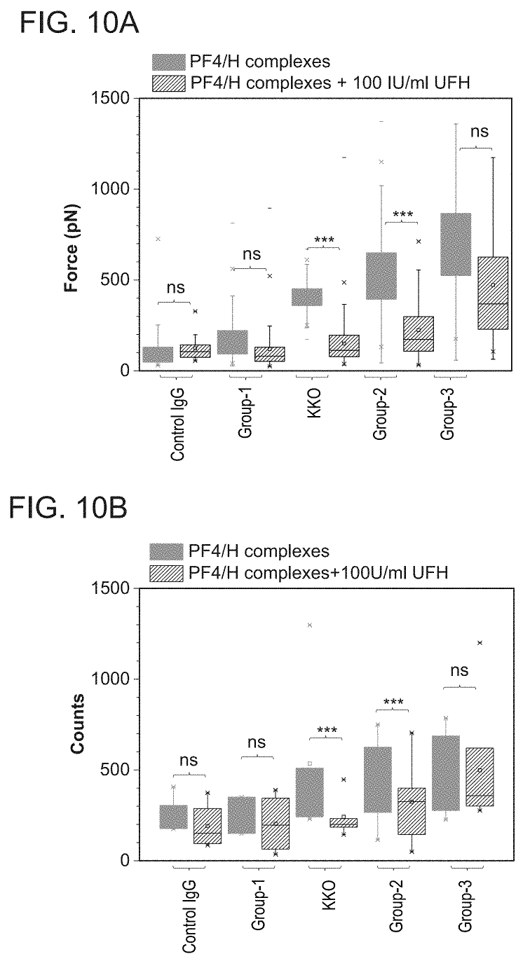

To achieve an optimal conformational change of PF4 for binding of anti-PF4/P Abs, platelets were coated with pre-formed PF4/H complexes (20 μg/ml PF4 and 0.5 U/ml UFH), known to provide optimal antibody reactivity in the EIA. This resulted in comparable binding forces (FIG. 5A-B) and a similar frequency of interactions (count numbers) (FIG. 5C) as when 50 μg/mL of PF4 had been coated. High heparin concentration (100 IU/ml) reduced the rupture forces and the rupture counts (FIG. 10).

### Epitope Exposure of PF4/H Complexes on Different Substrates

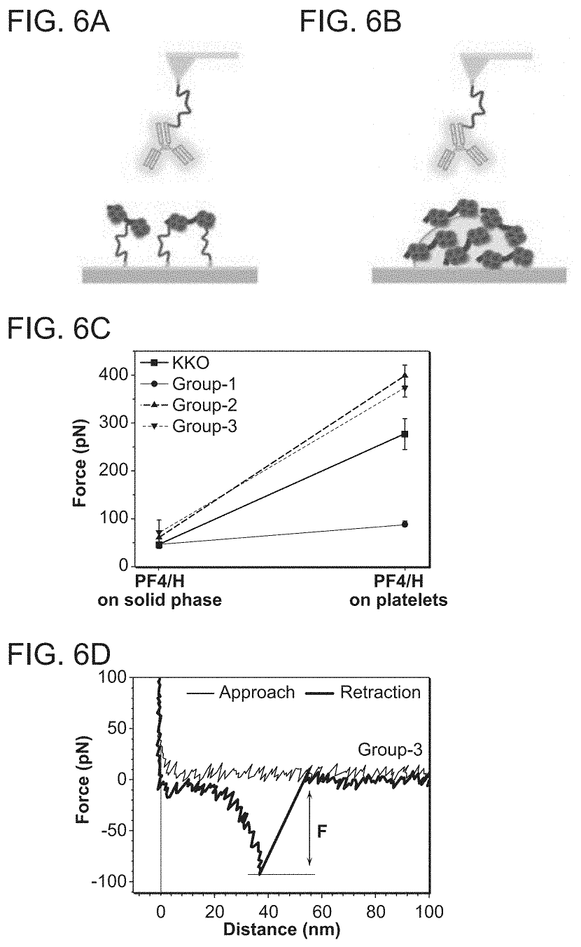

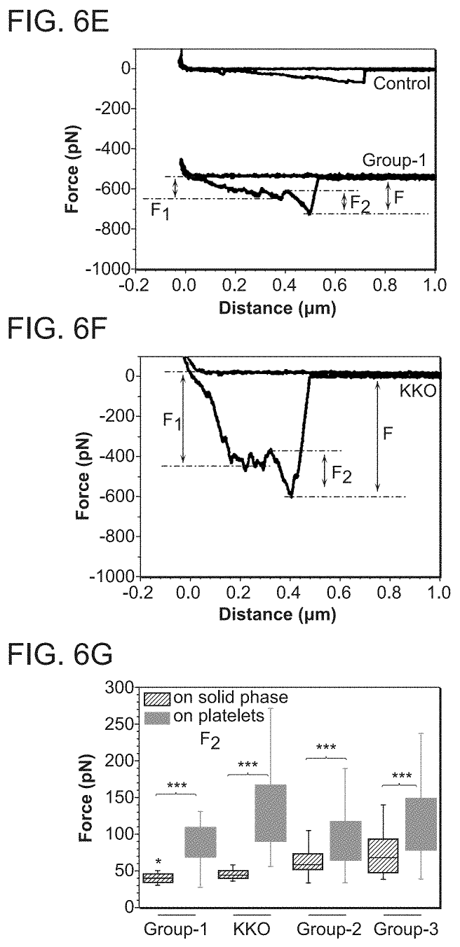

Previously, different binding forces of KKO to PF4/H complexes was observed depending on whether PF4/H complexes were immobilized on a solid phase or directly on platelet surfaces. To examine if human anti-PF4/P Abs show a similar behavior as the mouse monoclonal antibody KKO, the interaction forces of the same antibodies were compared when interacting with PF4/H complexes immobilized either on a solid phase (FIG. 6A) or on platelet surfaces (FIG. 6B). Average rupture forces of the same KKO, group-2, and group-3 Abs were significantly higher when interacting with PF4/H complexes immobilized on platelets than with PF4/H complexes immobilized on the solid phase (FIG. 6C), while the binding strength increased only marginally for group-1 Abs (FIG. 6D).

In contrast to the purified system with PF4/H complexes coated to a gold surface, binding forces for all antibodies increased, including the binding forces of control antibodies. Therefore, the individual force-distance curves were closely analyzed and it was found that they are a composite of several forces (F=F₁+F₂), where F₂ is the final rupture force representing the rupture force between antibody and antigen. The other rupture forces (F₁) developed gradually in a saw tooth-like structure. Without intending to be bound by theory, F₁ most likely reflects stretching of the antigen away from the platelet surface with complex multiple interactions of the platelet membrane, the platelet cytoskeleton, or extension of the component to which PF4 has bound, e.g. a glycoprotein (FIG. 6E-F). These F₂ forces were still significantly higher for KKO, group-2, and group-3 Abs binding to the platelet surface, than the forces measured when PF4/H complexes were coated on a gold surface. Control IgG only showed weak F₁ and F₂ forces, indicating weak interactions of antibodies with the platelet surface. Hereby the F₂ force reflects the force of unspecific control IgG binding (or attachment) to the platelet surface.

### DISCUSSION

Different physical characteristics of clinically relevant, platelet activating and non-relevant (non-activating) anti-PF4/P antibodies is provided herein, which provides a basis for diagnostic assays that differentiate between these antibody groups.

Non-platelet activating antibodies (group-1) show weak binding forces, which are not enhanced by adding PF4 or PF4/H complexes to the platelet surface. Platelet-activating, but heparin-dependent antibodies (group-2), show increased binding forces when platelets are pre-coated with PF4. Antibodies which activate platelets independently of heparin (group-3), isolated from sera of patients with autoimmune HIT, bind strongly to platelets even in the absence of PF4, but the number of binding counts is increased when PF4 is added.

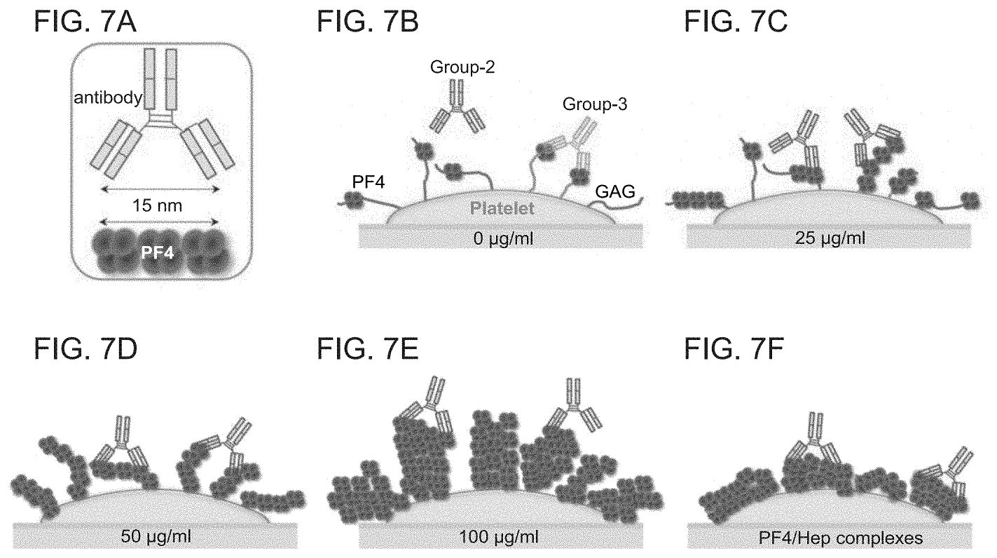

These findings have additional implications: They further underscore the high relevance of functional assays to detect clinically relevant, platelet activating antibodies. In contrast to antigen tests, non-activating PF4/P Abs hardly interact with platelets. They further underscore that PF4 can bind to polyanions on the platelet surface, which induces binding sites for anti-PF4/P Abs. A bell-shaped dependency of antibody binding forces related to the added PF4 concentration was observed. This is consistent with the previously described higher sensitivity of functional assays for PF4/P Abs when PF4 is added to platelets. While the peak of interaction was reached at 50 μg/mL PF4, both binding forces and binding counts decreased at lower and higher concentrations. Single-molecule studies provide an explanation for these observations. Without intending to be bound by theory, molecules with the same charge typically keep a certain distance from each other due to their repelling zeta potential. It was previously shown that the binding epitope of PF4/P Abs is exposed when 2 PF4 molecules come into close approximation, allowing the charge cloud to fuse. This provides the energy inducing the conformational change of PF4 needed to expose the antibody binding epitope(s). When no external PF4 is added, endogenous platelet PF4 is expressed on the platelet surface, but does not form complexes exposing a neoepitope. Most likely in this situation the density of PF4 on the platelet surface is too low. Without adding PF4 (FIG. 7B), few PF4 molecules bind to the platelet surface. They do not form complexes which only allows binding of autoimmune Abs (group-3). This is demonstrated herein by the fact that only the heparin independent group-3 Abs bind strongly to platelets but not heparin dependent group-2 Abs or KKO.

The distance between two binding sites of the Fab arms of an antibody is ˜15 nm, while the PF4 tetramer has a diameter of ˜5 nm². This means that minimal three PF4 molecules need to be aligned on a GAG in order to allow binding of both Fab arms of an IgG molecule to the same multimolecular complex (FIG. 7A). Although it has been shown that PF4 molecules are merged closely to each other when they form a complex with heparin, this likely does not change the total diameter of the single PF4 tetramer considerably. In addition, according to the model of PF4/H complexes provided by x-ray crystallography, PF4 molecules align staggered around the polyanion chain, which would make it also difficult for one antibody to bind with both Fab arms to two adjacent PF4 molecules.

When 25 μg/mL PF4 are added (FIG. 7C), some PF4/P complexes are formed but antibody binding is still very variable (FIG. 9 and FIG. 3-4). At this concentration, only a few large PF4/P complexes are formed and small PF4 complexes (likely consisting of only two PF4 tetramers per GAG) are randomly distributed on the platelet surface likely too far apart from each other to allow binding of both arms of group-2 IgG molecules. When only one arm of the PF4/H Abs binds, the resulting binding force is much weaker compared to binding of both arms. At 50 μg/mL (FIG. 7D), however, several PF4 molecules bind rather close to each other on the platelet surface allowing binding of both Fab arms of an anti-PF4/P antibody. As a result, highest binding forces (FIG. 3-4) and less variation (FIG. 9) were obtained at this concentration. This model is further corroborated by the experiment showing no difference in binding strength of PF4/P Abs to platelets pre-coated with PF4/H complexes or platelets precoated with 50 μg/mL PF4 (FIG. 7F). At higher PF4 concentrations (100 μg/ml) (FIG. 7E), likely too many PF4 molecules compete for the polyanions on the platelet surface creating several layers with less optimal antigen exposure, an effect resembling the prozone effect in immunohematology in the presence of very high concentrations of antibodies competing for an antigen. This again blocks optimal access of PF4/P Abs to their binding epitope. Consequently, the binding forces of all antibodies, including group-3 Abs, was reduced at 100 μg/ml PF4 (FIG. 3, FIG. 4E, FIG. 9). This is further supported by the flow cytometry measurements which show even higher PF4 binding to platelets at 100 μg/ml PF4 (FIG. 8), further indicating that reduced binding forces are rather caused by less optimal binding of group-2 Abs and KKO, or in case of group-3 Abs by weaker binding of PF4 to the platelet surface.

In clinical situations empirical observations show that with increased platelet activation resulting in increased PF4 release, e.g. after major surgery, HIT is more frequent than in situations with less platelet activation, and findings in the laboratory show that platelets expressing a higher number of PF4 molecules are more sensitive in interacting with anti-PF4/P Abs than platelets expressing lower amounts of PF4.

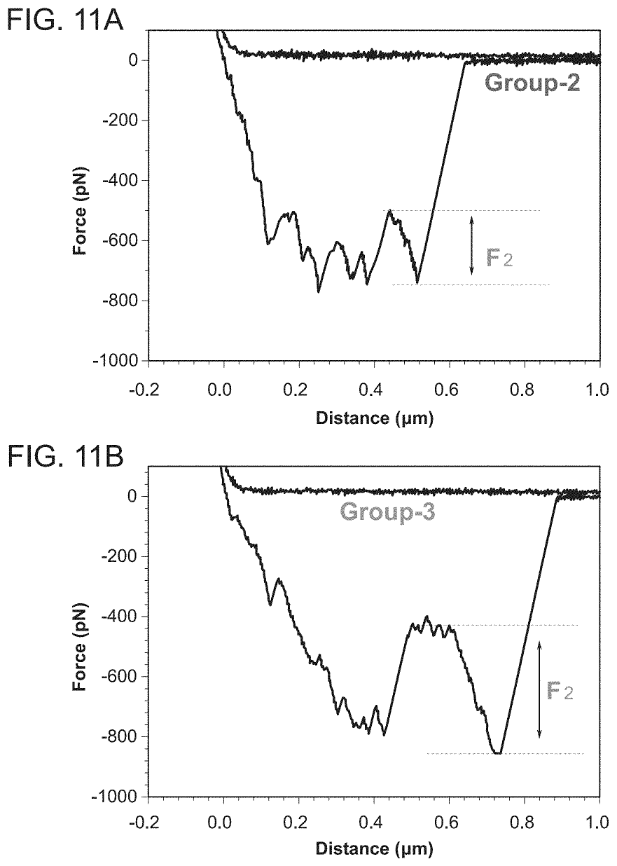

Binding forces of all antibody groups were enhanced when tested on the platelet surfaces as compared to the purified system. Differences were less pronounced for control IgG and group-1 Abs. As shown by the force curves of control IgG, even normal IgG interacts weakly with the platelet membrane. The force-distance curves of control IgG and anti-PF4/P Abs showed differences. The final peak which represents the unbinding force, when the antibody ruptures from its antigen, was minimal for control IgG, most likely reflecting non-specific interactions of IgG with the platelet membrane. Therefore, only the final rupture force (F₂) when PF4/P Abs interacted with PF4 coated platelets was compared to the total rupture force F when the same antibody interacted with PF4/H complexes on the solid phase. This again showed higher rupture forces F₂ further indicating that antibody binding to PF4/P complexes on the platelet surface differs from antibody binding to PF4/H complexes on the solid phase. Without intending to be bound by theory, it is likely that the three-dimensional orientation of PF4/P complexes is important for optimal antibody binding. The F₁ forces of KKO, group-2, and group-3 Abs most likely result from a series of complex interactions, including: pulling the complex of antibody, PF4, and GAG from the platelet membrane; stretching of the GAG chain and stretching the super-soft platelet membrane before the antibody ruptures from its antigen. If the binding force is weak (e.g. group-1 Abs), the final rupture occurs early and fewer sub-factors are involved, whereas strong binding forces (e.g. group-2 and group-3 Abs) leads to ‘a delay of rupture’ and the F₁ force is a composite of multiple factors involved in the pulling process (FIG. 11). In addition, as shown by the rupture force of control IgG some non-specific interactions between antibodies and the platelet membrane may occur which can be sensitively detected by SMFS.

In summary, these findings disclosed herein characterize the interaction of PF4/P antibodies with platelets and provide an explanation of why in clinical situations with increased PF4 release, patients have a higher risk of developing heparin-induced thrombocytopenia. The clear difference in binding between non-platelet activating antibodies and platelet activating antibodies when interacting with PF4 bound to polyanions on the platelet surfaces compared to PF4/P complexes immobilized on the solid phase is the basis for specific antigen assays for the detection of clinically relevant HIT antibodies as disclosed herein.

While this disclosure has been particularly shown and described with references to examples thereof, it will be understood by those skilled in the art that various changes in form and details may be made therein without departing from the spirit and scope of the present application as defined by the appended claims. Such variations are intended to be covered by the scope of this present application. As such, the foregoing description of examples of the present application is not intended to be limiting, the full scope rather being conveyed by the appended claims.

What is claimed is:

1. A method of identifying platelet activating anti-PF4/P antibodies in a biological sample comprising:
a) providing PF4-coated or PF4/P-coated biological membranes or PF4-coated or PF4/P coated artificial membranes;
b) contacting the PF4-coated or PF4/H-coated membranes with a biological sample from a patient in the presence of conditions whereby platelet-activating antibodies bind the coated membranes and non-activating antibodies do not bind the coated membranes; and
c) detecting antibodies bound to the coated membranes.

2. The method of claim 1, wherein said biological membranes comprise whole cells.

3. The method of claim 1, wherein said biological membranes comprise cell fractions.

4. The method of claim 3, wherein said cell fractions comprise platelet microparticles.

5. The method of claim 1, wherein said biological or artificial membranes are coated onto a solid substrate.

6. The method of claim 1, wherein said artificial membranes comprise phospholipids and PF4-binding molecules and optionally heparin-binding molecules.

7. The method of claim 6, wherein said PF4-binding molecules and heparin-binding molecules comprise proteins, carbohydrates, nucleic acids or polymers.

8. The method of claim 7, wherein said proteins comprise PF4 antibodies or fragments thereof, heparin antibodies or fragments thereof, FcγRIIa receptors or fragments thereof, or protamines or fragments thereof.

9. The method of claim 7, wherein said nucleic acids comprise affimers that specifically bind PF4 or heparin.

10. The method of claim 7, wherein said polymers comprise polyanionic polymers.

11. The method of claim 10, wherein said polyanionic polymer is selected from the group consisting of: polyvinyl sulfate, polystyrene sulfate, chondroitin sulfate, dermatan sulfate, keratan sulfate, heparan sulfate and heparin.

12. The method of claim 1, wherein said conditions comprise pH and ion concentration.

13. The method of claim 1, further comprising a step between steps b) and c) of washing the membranes with a solution comprising a pH and ion concentration such that platelet-activating antibodies remain bound and non-activating antibodies are removed.

14. The method of claim 12, wherein said pH is about 6.0 and said ion concentration is about 50 mM NaCl.

15. The method of claim 1, wherein said PF4-coated membranes are formed by incubating with 2-500 μg/ml PF4.

16. The method of claim 15, wherein said incubating is at 37° C. for about 30 minutes.

17. The method of claim 1, wherein said PF4/P-coated membranes are formed by incubating with 2-500 μg/ml of PF4/P pre-formed by PF4 and a polyanionic polymer.

18. The method of claim 17, wherein said polyanionic polymer is selected from the group consisting of: polyvinyl sulfate, polystyrene sulfate, chondroitin sulfate, dermatan sulfate, keratan sulfate, heparan sulfate and heparin.

19. The method of claim 18, wherein said polyanionic polymer is polyvinyl sulfate.

20. The method of claim 18, wherein said polyanionic polymer is heparin.

21. The method of claim 17, wherein said incubating is at 37° C. for about 30 minutes.

22. The method of claim 5, wherein said solid substrate is selected from the group consisting of: glass surfaces, plastic surfaces, silicon surfaces, solid organic polymers, cellulose/cellulose-based membranes, colloidal metal particles and magnetic particles.

23. The method of claim 22, wherein said solid substrate is magnetic particles.

24. The method of claim 22, wherein said solid substrate is a glass surface comprising a glass slide.

25. The method of claim 22, wherein said solid substrate is a plastic surface comprising a microtiter plate.

26. The method of claim 22, wherein said solid substrate is a colloidal metal particle comprising a gold particle.

27. The method of claim 22, wherein said solid substrate is a solid organic polymer comprising a latex bead.

28. The method of claim 1, wherein said biological sample is selected from the group consisting of: a blood sample, a serum sample and a plasma sample.

29. The method of claim 28, wherein said biological sample is a serum sample.

30. The method of claim 1, wherein said detecting is carried out by an assay selected from the group consisting of: an enzyme linked immunosorbent assay (ELISA), a radioimmunoassay (MA), an immuno radiometric assay (IRMA), a fluorescent immunoassay (FIA), a chemiluminescent immunoassay (CLIA), an electro-chemiluminescent immunoassay (ECL) and an agglutination assay.

31.-90. (canceled)
